(19) United States
(12) Patent Application Publication
(10) Pub. No.: US 2013/0286536 A1
(43) Pub. Date: Oct. 31, 2013

(54) IMPLANTABLE MEDICAL DEVICE WITH FEEDTHROUGH, FEEDTHROUGH AND METHOD

(71) Applicant: MEDTRONIC, INC., Minneapolis, MN (US)

(72) Inventors: Rajesh V. Iyer, Eden Prairie, MN (US); Lea A. Nygren, Bloomington, MN (US); Brad C. Tischendorf, Minneapolis, MN (US)

(73) Assignee: MEDTRONIC, INC., Minneapolis, MN (US)

(21) Appl. No.: 13/772,465

(22) Filed: Feb. 21, 2013

Related U.S. Application Data
(60) Provisional application No. 61/638,775, filed on Apr. 26, 2012.

(51) Int. Cl. A61N 1/05

(52) U.S. Cl. CPC A61N 1/05
USPC 361/302; 29/825

(57) ABSTRACT

Feedthrough and method for making a feedthrough. The feedthrough has a ferrule forming a ferrule lumen, an electrically conductive pin extending longitudinally through at least a portion of the ferrule lumen, a filter capacitor surrounding the electrically conductive pin within the ferrule lumen, the filter capacitor having a bonding surface, and a ceramic seal positioned within the ferrule lumen directly abutting the filter capacitor sealing a space between the electrically conductive pin and the ferrule. The ceramic seal adheres to and creates an adhesive bond with the bonding surface of the capacitor and substantially inhibits fluid flow through the ferrule lumen.

### FIELD

The present invention relates generally to implantable medical devices and, more particularly, to implantable medical devices having a feedthrough, a feedthrough for an implantable medical device and methods for making such feedthroughs.

### BACKGROUND

Implantable medical devices which deliver electrical stimulation to patient tissue, such as pacemakers, defibrillators and neurological stimulators, need to be able to transmit electrical pulses from electronic circuits within the implantable medical device while at the same time inhibiting bodily fluids from entering the implantable medical device and substances from leaving the implantable medical device to the extent possible. Electrical feedthroughs are commonly configured on such implantable medical devices to provide for the transmission of electrical pulses while also maintaining substantial or complete fluid isolation between the interior of the implantable medical device and the patient. Other implantable medical devices may utilize feedthroughs for other purposes providing an electrical ingress or egress to or from the implantable medical device or providing a throughput for some other therapeutic or diagnostic function. In addition or alternatively, a feedthrough may incorporate a capacitor or filter assembly to reduce an amount of electromagnetic interference which may enter the implantable device.

Historically, implantable medical device feedthroughs have utilized a ferrule extending through a housing of the implantable medical device. An electrically conductive wire or pin is positioned within and extending through the ferrule. Various seals and electronic devices are then positioned around the pin and bonded to the pin and the ferrule to provide isolation. Such seals have typically incorporated an insulative bulk or device and gold and solder preforms to bond the insulative device to the pin and the ferrule. While the gold has provided bonding while also being substantially biocompatible, solder provides bonding which is inexpensive and easy to manipulate.

However, such feedthrough structures are relatively complex to manufacture. The different materials utilized need to be positioned with considerable precision. The use of both gold and solder to bond the insulative device and other electronic components, such as capacitors, to the pin and the ferrule compels multiple bonding steps at different temperatures, owing to the practice of heating each material to a different temperature to cause the material to melt and flow, while also preventing overheating of the other materials. In addition, the different steps may create more opportunities for a fault to occur, resulting either in corrective action or disposal of the feedthrough.

### SUMMARY

An implantable medical device and feedthrough, and method for making such feedthrough, has been developed which addresses these challenges by reducing the complexity of the feedthrough through the use of relatively non-conventional materials. Instead of utilizing an insulative bulk, gold and solder, the feedthrough described herein utilizes a ceramic seal in place of the insulative bulk and an active braze alloy to bond a capacitor to the pin and ferrule. Glass advantageously functions as a ceramic which, when heat is applied and progressively increases, softens and begins to flow without melting. As part of the softening of the glass as the temperature increases, the glass develops contact with the pin and ferrule which, upon cooling, creates a bonded seal between the pin and ferrule.

Active braze alloys are manufacturable compounds of different materials. Depending on the mix of materials in the particular active braze alloy selected, the active braze alloy may be selected to melt at particular temperatures. The selectability of the active braze alloy, combined with the softening characteristics of the glass over temperature increases, provides for a simplified manufacturing process, and in particular one with a single heating step. The feedthroughs disclosed herein may be manufactured by applying heat to the feedthrough assembly so as to begin softening the ceramic or glass, and then gradually increasing the applied temperature. As the temperature increases, the ceramic or glass softens and contacts the pin and ferrule. The temperature is increased until a desired temperature below the melting point of the ceramic or glass is reached and the bonding characteristics of the ceramic or glass upon cooling is achieved. The active braze alloy is selected to have a melting or liquidus temperature somewhat below the final temperature applied to the feedthrough. In so doing, the ceramic or glass may be softened and fill the space between the pin and the ferrule as desired, whereupon the active braze alloy may melt and bond the capacitor to the pin and ferrule. Thus, a single temperature sweep may soften and bond the ceramic or glass and melt and bond the active braze alloy.

In an embodiment, a feedthrough comprises a ferrule forming a ferrule lumen, an electrically conductive pin extending longitudinally through at least a portion of the ferrule lumen, a filter capacitor surrounding the electrically conductive pin within the ferrule lumen, the filter capacitor having a bonding surface, and a ceramic seal positioned within the ferrule lumen directly abutting the filter capacitor sealing a space between the electrically conductive pin and the ferrule. The ceramic seal adheres to and creates an adhesive bond with the bonding surface of the capacitor and substantially inhibits fluid flow through the ferrule lumen.

In an embodiment, the bonding surface of the capacitor is substantially non-metallic.

In an embodiment, the ceramic seal is a glass seal.

In an embodiment, the ceramic seal substantially covers the bonding surface of the filter capacitor abutting the ceramic seal to create the adhesive bond.

In an embodiment, the ceramic seal substantially covers the bonding surface by wetting out, at least in part, the bonding surface of the filter capacitor.

In an embodiment, the ceramic seal substantially surrounds the electrically conductive pin.

In an embodiment, the feedthrough further comprises a preform operatively electrically coupling the capacitor to the pin.

In an embodiment, the preform comprises a ring of an active braze alloy.

In an embodiment, the ceramic seal has a softening temperature and a ceramic melting temperature greater than the softening temperature, and the preform has a preform melting temperature greater than the softening temperature and less than the ceramic melting temperature.

In an embodiment, the softening temperature is approximately 735 degrees Celsius and the preform melting temperature is approximately 810 degrees Celsius.

In an embodiment, the preform comprises a first active braze alloy ring operatively electrically coupling the filter capacitor to the electrically conductive pin and a second active braze alloy ring operatively electrically coupling the capacitor to the ferrule.

In an embodiment, the filter capacitor has a coating configured to electrically couple with the preform.

In an embodiment, the filter capacitor is oriented interior of the feedthrough and wherein the ceramic seal is oriented exterior of the feedthrough, relative to each other.

In an embodiment, an implantable medical device comprises a housing forming a plurality of feedthrough openings and a plurality of feedthroughs, each individually positioned within one of the feedthrough openings. Each feedthrough comprises a ferrule forming a ferrule lumen, an electrically conductive pin extending longitudinally through at least a portion of the ferrule lumen, a filter capacitor surrounding the electrically conductive pin within the ferrule lumen, the filter capacitor having a bonding surface and a ceramic seal positioned within the ferrule lumen directly abutting the filter capacitor sealing a space between the electrically conductive pin and the ferrule. The ceramic seal adheres to and creates an adhesive bond with the bonding surface of the capacitor and substantially inhibits fluid flow through the ferrule lumen;

In an embodiment, the plurality of feedthroughs are spaced on centers not more than 0.889 millimeters apart.

In an embodiment, a method for making a feedthrough, comprises the steps of positioning an electrically conductive pin longitudinally through at least a portion of a ferrule lumen of a ferrule, positioning a filter capacitor surrounding the electrically conductive pin within the ferrule lumen, and positioning a preform proximate the filter capacitor, positioning a ceramic seal within the ferrule lumen and directly abutting the filter capacitor. Then an ambient temperature is progressively increased from a first temperature at least as low as a softening temperature of the ceramic seal to a second temperature at least as high as a preform melting temperature of the preform so that the ceramic seal softens and substantially occupies the ferrule lumen between the ferrule and the electrically conductive pin before the preform melts and operatively electrically couples the filter capacitor to the electrically conductive pin.

In an embodiment, the progressively increasing the ambient temperature step causes the ceramic seal to substantially cover the bonding surface of the filter capacitor abutting the ceramic seal to create the adhesive bond.

In an embodiment, the progressively increasing the ambient temperature step causes the ceramic seal to substantially cover the bonding surface of the filter capacitor by wetting out, at least in part, the bonding surface of the filter capacitor.

In an embodiment, the positioning the ceramic seal step substantially surrounds the electrically conductive pin with the ceramic seal.

In an embodiment, the progressively increasing the ambient temperature step increases the ambient temperature to approximately 850 degrees Celsius.

In an embodiment, the preform comprises a first active braze alloy ring and a second active braze alloy ring and the positioning the preform step comprises positioning the first active braze alloy ring proximate the electrically conductive pin and positioning the second active braze alloy ring proximate the ferrule.

### FIGURES

FIG. 1 is an abstract, cross-sectional drawing of an electrical feedthrough;

FIG. 2 is an abstract depiction of an implantable medical device incorporating multiple feedthroughs of FIG. 1; and

FIG. 3 is a flowchart for making a feedthrough as in FIG. 1.

### DESCRIPTION

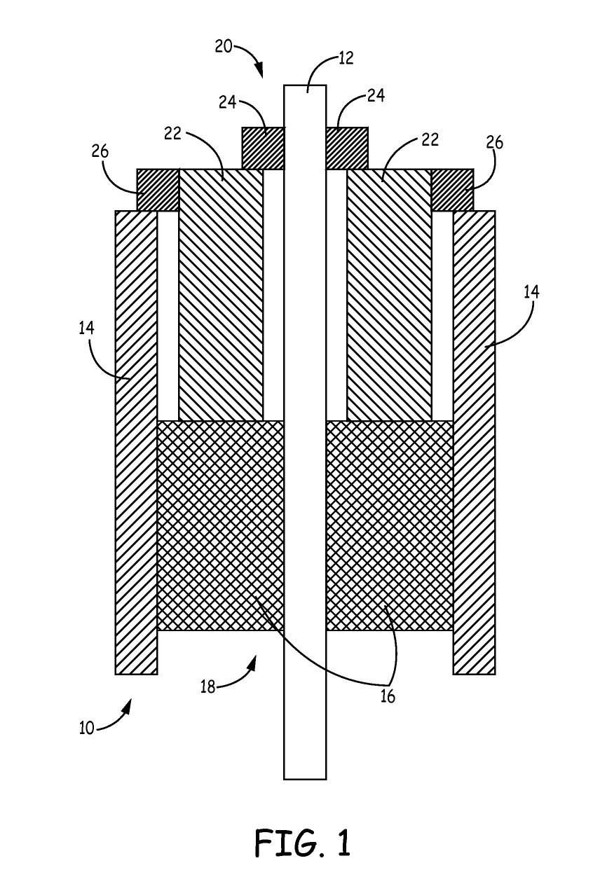

FIG. 1 is an abstract, cross-sectional drawing of feedthrough 10. Electrically conductive pin 12 extends through ferrule 14. In various embodiments, pin 12 is comprised of a biocompatible metal, such as titanium, niobium or other metal, including certain precious metals.

Ceramic seal 16 is selectable from various standard types of glass, glass-ceramics or ceramics generally. It is to be recognized and understood that the term “ceramic seal” as used herein encompasses both a seal made from a ceramic material, a glass material, a “glass-ceramic” material or mixtures thereof Generally, a ceramic material is any inorganic, nonmetallic solid. A glass material can be generally described as a ceramic which is not crystalline. A glass-ceramic material is a glass material which has been processed to have a limited crystalline structure, sometimes described as having relatively small, localized crystals, or has been blended with a crystalline ceramic.

Various glasses which may be utilized include, but are not limited to, alkaline-earth aluminoborates (disclosed in U.S. Pat. No. 6,090,503, which is incorporated by reference in its entirety), lanthanum alumino-borates (disclosed in U.S. Pat. No. 8,129,622, which is incorporated herein by reference in its entirety) and boro-alumino-silicates (disclosed in U.S. Pat. Nos. 5,866,851 and 5,294,241, which are incorporated by reference in their entirety). In an embodiment, ceramic seal 16 is made from a lanthanum alumino-borate glass. Upon the completion of the manufacturing process described herein, ceramic seal 16 may provide at least partial isolation between first side 18 and second side 20 of feedthrough 10. In an embodiment, first side 18 is an exterior side of feedthrough 10 configured to be in contact with biological material and fluid while second side 20 is an interior side of feedthrough 10 not necessarily configured to contact with biological material and fluid.

Capacitor 22 may provide some protection against certain changes in environmental electromagnetic conditions, including electromagnetic fields generated by external sources. In an embodiment, capacitor is in various alternative embodiments, capacitor 22 may be substituted with or included in addition to additional filter capacitors and inductors. Preforms 24, 26 may be positioned with respect to pin 12, ferrule 14 and capacitor 22 to physically secure such components with respect to one another upon preforms 24, 26 having been heated to a predetermined melting or liquidus temperature. In various embodiments, preforms 24, 26 are made from an active braze alloy. In various embodiments, the active braze alloy is Cusil-ABA, or Cusil active braze alloy, a brand name for an alloy of 63% silver, 35.25% copper and 1.75% titanium, or Cusin-ABA, Cusin active braze alloy, a brand name for an alloy of 63% silver, 34.25% copper, 1% tin and 1.75% titanium.

In various embodiments, the ceramic glass of ceramic seal 16 may bond with or “wet out” capacitor 22 as well as pin 12 and ferrule 14. Bonding of ceramic seal 16 with capacitor 22 may reduce a likelihood of surface breakdown between ceramic seal 16 and capacitor 22 as a result of relatively high voltage inputs to feedthrough 10, such as a cardioversion or defibrillation shock. In various embodiments in which ceramic seal 16 is a ceramic material, capacitor 22 is not and does not need to be metalized to promote bonding with ceramic seal 16. Consequently, a need for a separate insulating material between ceramic seal 16 and capacitor 22 may be obviated. Similarly, in various embodiments, preforms 24, 26 are comprised of materials which may bond with pin 12, ferrule 14 and capacitor 22 without need for metallization of those components. It is noted that pin 12, ferrule and capacitor 22 may be metalized to further promote bonding, but that such metalizing may not be necessary.

Because active braze alloys are, as known in the art, configurable at the time of manufacture based on the materials which are utilized to make the alloy, the active braze alloy actually used to create certain embodiments of preforms 24, 26 may be formed with a selectable melting temperature. In an embodiment an active braze alloy is Cusil having a liquidous temperature of approximately eight hundred ten (810) degrees Celsius. In various alternative embodiments, active braze alloys with various different melting temperatures are utilized instead. In various embodiments, feedthrough 10 is heated to approximately thirty (30) degrees Celsius greater than the melting temperature of the selected active braze alloy. In the above embodiment, feedthrough 10 is heated to at least eight hundred fifty (850) degrees Celsius. Upon being heated to the melting or liquidus temperature of the active braze alloy, preforms 24, 26 soften and flow, with molecules of preforms 24, 26 potentially chemically bonding to other adjacent molecules.

In various embodiments, ceramic seal 16 is selected and configured so that the thermal characteristics of the material of ceramic seal 16 provide for ceramic seal 16 to soften and begin to flow at a temperature less than the melting temperature of the selected active braze alloy but which has a melting temperature greater than the melting temperature of the active braze alloy. Alternatively, the material of preforms 24, 26 may be selected based on the selection of ceramic seal 16 such that preforms 24, 26 have a melting temperature greater than the softening temperature of ceramic seal 16 but less than the melting temperature of ceramic seal 16. In various embodiments, ceramic seal 16 begins to soften at less than eight hundred (800) degrees Celsius and begins to melt at not less than one thousand (1000) degrees Celsius. In the above embodiment, ceramic seal 16 is a lanthanum alumino-borate glass having a softening temperature of seven hundred thirty-five (735) degrees Celsius and a melting temperature of greater than one thousand eight hundred one thousand (1000) degrees Celsius.

Feedthrough 10 may be constructed by positioning pin 12 within ferrule 10 and then positioning ceramic seal 16, capacitor 22 and preforms 24, 26 with respect to pin 12 and ferrule 14 as shown in FIG. 1. Feedthrough 10 may then be heated by placing feedthrough 10 in an environment having a first temperature and increasing the temperature from the first temperature though the softening temperature of ceramic seal 16 and at least to the melting temperature of active braze alloy rings 24, 26 or, in the above embodiment, eight hundred ten (810) degrees Celsius but less than the melting temperature of ceramic seal 16.

The use of ceramic seal 16 in combination with preforms 24, 26 results in a heating process that may use only a simple temperature ramp, although in embodiments, multiple temperatures and temperature ramps may also be used. As the temperature reaches and exceeds the softening temperature of ceramic seal 16, ceramic seal 16 begins to deform and make contact fits with at least pin 12 and ferrule 14. Ceramic seal 16 may bond with pin 12 and ferrule 14 and at least partially inhibit the flow of fluid and gas though ferrule 14. As the temperature increases, ceramic seal 16 may become softer, improving contact and bonding with pin 12 and ferrule 14, but due to the viscous nature of ceramic seal 16, ceramic seal 16 may not melt and lose structural continuity.

Eventually, the temperature ramp reaches and passes through the melting temperature of preforms 24, 26, causing the rings to melt, flow and bond with ferrule 14 and capacitor 22. With both ceramic seal 16 and preforms 24, 26 bonded, the temperature ramp may be stopped and ferrule 10 may be cooled with both ceramic seal 16 and preforms 24, 26 providing at least partial sealing between first side 18 and second side 20 of feedthrough 10.

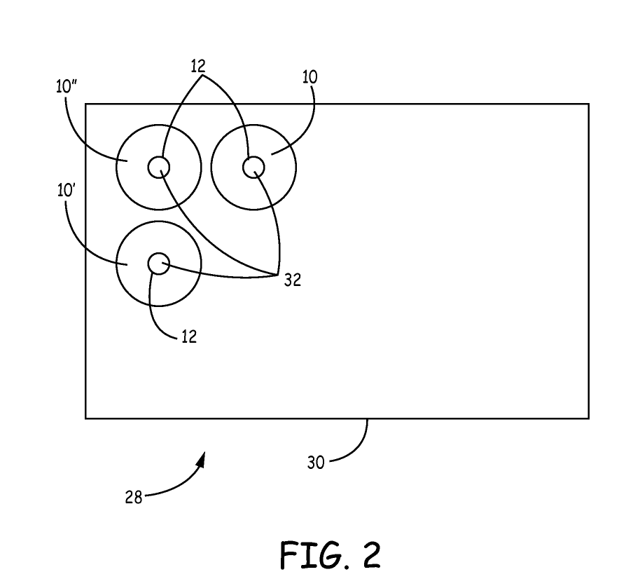

FIG. 2 is a diagrammatic illustration of multiple feedthroughs 10 on an exemplary implantable medical device 28. In various embodiments, implantable medical device 28 has electronics for monitoring a patient condition and delivering an electrical therapeutic output. Such implantable medical devices 28 may include, but are not limited to, pacemakers, cardioverter/defibrillators and neurological stimulators. Implantable medical device 28 includes housing 30 enclosing certain electronics of implantable medical device 28. In various embodiments, housing 30 is substantially sealed for implantation in a patient. In an embodiment housing 30 is hermetically sealed. Feedthroughs 10 provide electrical connectivity between an object external to housing 30, such as a medical lead, and electronics of implantable medical device 28 contained within housing 30.

In various embodiments, centers 32 of pins 12 of adjacent feedthroughs 10 are separated by a predetermined distance. The predetermined distance may be minimized to promote housing 30 and implantable medical device 28 being relatively small. Advantageous use of the teachings herein may allow an implantable medical device having a plurality of feedthroughs spaced more closely together than may otherwise have been realistically feasible. For example, an implantable medical device may be constructed with a plurality of feedthroughs spaced at least as close to each other as being on 0.035 inch/0.889 millimeter centers. In such an embodiment, centers 32 of pins 12 of adjacent feedthroughs 10′, 10″ are separated by approximately 0.035 inches/0.889 millimeters. In an example of use of such an embodiment, feedthroughs 10 may be configured to pass relatively low voltage current, such as may be utilized by a pacemaker or neurological stimulator to deliver conventional pulses of generally not more than ten (10) Volts in amplitude. While relatively close center-to-center spacing of feedthroughs may be desirable, it is also contemplated that in an embodiment, centers 32 of pins 12 of adjacent feedthroughs 10′, 10″ may be separated by approximately 0.055 inches/1.397 millimeters. In such an embodiment, feedthroughs 10 may be configured to pass relatively high voltage current, such as may be utilized by a cardioverter/defibrillator to deliver conventional cardioversion/defibrillation pulses. In such embodiments, such spacing may be an approximately thirty (30) percent or greater improvement over a conventional feedthrough known in the art.

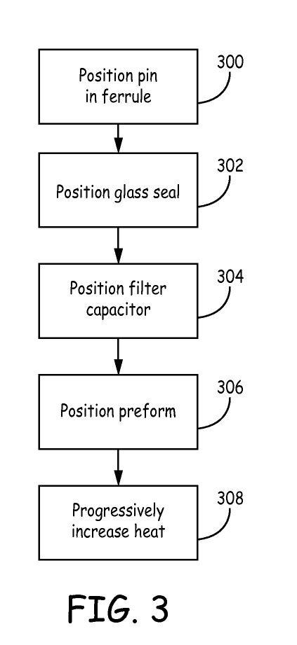

FIG. 3 is a flowchart for making feedthrough 10. Pin 12 is positioned (300) longitudinally within the lumen of ferrule 14. Ceramic seal 16 is positioned (302) within lumen 14 and directly abutting filter capacitor 22. Filter capacitor 22 is positioned (304) surrounding pin 12 within ferrule 14. Preforms 24, 26 are positioned (306) proximate filter capacitor 22. In various embodiments, preform 24 is positioned proximate filter capacitor 22 and pin 12 while preform 26 is positioned proximate filter capacitor 22 and ferrule 14. It is noted that positioning (300, 302, 304, 306) the various components may occur in any order and that the above recitation does not limit positioning the components to any particular sequence. In an embodiment, pin 12 is positioned (300), then ceramic seal 16 is positioned (302), then capacitor 22 is positioned (304), then preforms 24, 26 are positioned (306). The ambient temperature is progressively increased (308) from a first temperature, through the softening temperature of ceramic seal 16 and the preform melting temperature of preforms 24, 26, and to a second temperature less than the ceramic melting temperature of ceramic seal 16.

As a consequence of the progressively increased temperature, ceramic seal 16 softens and at least partially seals feedthrough 10 by bonding, at least in part, with at least some of pin 12 and lumen 14. Additionally, ceramic seal 16 at least partially wets out and bonds with filter capacitor 22 to reduce a risk of surface arcing between ceramic seal 16 and filter capacitor 22. As a further consequence of the progressive increase in temperature, preforms 24, 26 melt and bond with pin 12, ferrule 14 and filter capacitor 22, at least partially securing and electrically connecting at least some of pin 12, ferrule 14 and filter capacitor 22 with respect to one another.

1. A feedthrough, comprising:
a ferrule forming a ferrule lumen;
an electrically conductive pin extending longitudinally through at least a portion of said ferrule lumen;
a filter capacitor surrounding said electrically conductive pin within said ferrule lumen, said filter capacitor having a bonding surface;
a ceramic seal positioned within said ferrule lumen directly abutting said filter capacitor sealing a space between said electrically conductive pin and said ferrule;
said ceramic seal adhering to and creating an adhesive bond with said bonding surface of said capacitor and substantially inhibiting fluid flow through said ferrule lumen.

2. The feedthrough of claim 1 wherein said bonding surface of said capacitor is substantially non-metallic.

3. The feedthrough of claim 2 wherein said ceramic seal is a glass seal.

4. The feedthrough of claim 2 wherein said ceramic seal substantially covers said bonding surface of said filter capacitor abutting said ceramic seal to create said adhesive bond.

5. The feedthrough of claim 2 wherein said ceramic seal substantially covers said bonding surface by wetting out, at least in part, said bonding surface of said filter capacitor.

6. The feedthrough of claim 1 wherein said ceramic seal substantially surrounds said electrically conductive pin.

7. The feedthrough of claim 1 further comprising a preform operatively electrically coupling said capacitor to said pin.

8. The feedthrough of claim 7 wherein said preform comprises a ring of an active braze alloy.

9. The feedthrough of claim 7 wherein said preform comprises:
a first active braze alloy ring operatively electrically coupling said filter capacitor to said electrically conductive pin, and
a second active braze alloy ring operatively electrically coupling said capacitor to said ferrule.

10. The feedthrough of claim 7 wherein said filter capacitor has a coating configured to electrically couple with said preform.

11. The feedthrough of claim 1 wherein said filter capacitor is oriented interior of said feedthrough and wherein said ceramic seal is oriented exterior of said feedthrough, relative to each other.

12. An implantable medical device, comprising:
a housing forming a plurality of feedthrough openings; and
a plurality of feedthroughs, each individually positioned within one of said feedthrough openings, each feedthrough comprising:
a ferrule forming a ferrule lumen;
an electrically conductive pin extending longitudinally through at least a portion of said ferrule lumen;
a filter capacitor surrounding said electrically conductive pin within said ferrule lumen, said filter capacitor having a bonding surface; and
a ceramic seal positioned within said ferrule lumen directly abutting said filter capacitor sealing a space between said electrically conductive pin and said ferrule;
said ceramic seal adhering to and creating an adhesive bond with said bonding surface of said capacitor and substantially inhibiting fluid flow through said ferrule lumen.

13. The implantable medical device as in claim 12 wherein said plurality of feedthroughs are spaced on centers not more than 0.889 millimeters apart.

14. The implantable medical device of claim 12 wherein said bonding surface of said capacitor is substantially non-metallic.

15. The implantable medical device of claim 14 wherein said ceramic seal substantially covers said bonding surface of said filter capacitor abutting said ceramic seal to create said adhesive bond.

16. The implantable medical device of claim 14 wherein said ceramic seal substantially covers said bonding surface by wetting out, at least in part, said bonding surface of said filter capacitor.

17. The implantable medical device of claim 12 wherein said ceramic seal substantially surrounds said electrically conductive pin.

18. The implantable medical device of claim 12 further comprising a preform operatively electrically coupling said capacitor to said pin.

19. A method for making a feedthrough, comprising the steps of:
positioning an electrically conductive pin longitudinally through at least a portion of a ferrule lumen of a ferrule;
positioning a filter capacitor surrounding said electrically conductive pin within said ferrule lumen;
positioning a preform proximate said filter capacitor;
positioning a ceramic seal within said ferrule lumen and directly abutting said filter capacitor; and then
progressively increasing an ambient temperature from a first temperature at least as low as a softening temperature of said ceramic seal to a second temperature at least as high as a preform melting temperature of said preform so that said ceramic seal softens and substantially occupies said ferrule lumen between said ferrule and said electrically conductive pin before said preform melts and operatively electrically couples said filter capacitor to said electrically conductive pin.

20. The method of claim 19 wherein said bonding surface of said capacitor is substantially non-metallic.
